(19) United States
(12) Patent Application Publication
(10) Pub. No.: US 2026/0327311 A1
(43) Pub. Date: Oct. 22, 2026

(54) PH-SENSITIVE ANTI-CTLA-4 ANTIBODIES AND USES THEREOF

(71) Applicants: OncoC4, Inc., Rockville, MD (US); University of Maryland, Baltimore, Baltimore, MD (US)

(72) Inventors: Yang Liu, Potomac, MD (US); Pan Zheng, Potomac, MD (US); Yan Zhang, Rockville, MD (US); Martin Devenport, Gaithersburg, MD (US)

(21) Appl. No.: 19/485,045

(22) PCT Filed: May 17, 2024
(86) PCT No.: PCT/US2024/029920

§ 371 Date: Nov. 17, 2025

Related U.S. Application Data
(60) Provisional application No. 63/502,839, filed on May 17, 2023.

(51) Int. Cl. C07K 16/28; A61K 47/18; A61K 47/22; A61K 47/26; A61P 35/00

(52) U.S. Cl. CPC C07K 16/2818; A61K 47/183; A61K 47/22; A61K 47/26; A61P 35/00

(57) ABSTRACT

Provided herein are pH-sensitive anti-CTLA-4 antibodies, including variants of Ipilimumab, and therapeutic uses thereof.

### FIELD OF THE INVENTION

The present invention relates to pH-sensitive anti-CTLA-4 antibodies, including variants of Ipilimumab, and therapeutic uses thereof.

### REFERENCE TO THE SEQUENCE LISTING

This application contains a Sequence Listing that has been submitted in XML format via Patent Center and is hereby incorporated by reference in its entirety. The file, created on May 7, 2024, is named Sequence_Listing_111005_0605_01PC00.xml, and is 42 kb in size.

### BACKGROUND OF THE INVENTION

Two major challenges of targeting CTLA-4 in cancer immunotherapy are to reduce the immune-related adverse events (irAE) and improve broad therapeutic efficacy. According to conventional thinking, tumor immunity and irAE triggered by anti-CTLA-4 antibodies are inseparable. CTLA-4 checkpoint blockade boosts tumor killing responses, which inevitably causes cross-reactive autoimmunity, thereby making irAE a necessary price to pay for successful anticancer immunity.

The commercial anti-CTLA-4 monoclonal antibody (mAb), Ipilimumab, is frequently accompanied by a wide variety of adverse events in clinical mono-therapy or combination therapy. In particular, combination therapy with Ipilimumab and Nivolumab (anti-PD-1) have led to more than 50% patients developing grade 3 and grade 4 irAE in both melanoma and NSCLC. Ipilimumab-associated irAEs include hematological abnormalities such as pure red cell aplasia and non-infection-related inflammatory damage to solid organs. While some low grade irAE such as colitis or dermatitis are manageable, high grade irAE, such as pneumonitis, hepatitis, neurologic events and myocarditis, can be serious and life threatening. Although Ipilimumab-treated patients who survive for three years showed no further decline in survival rate over a ten-year period compared to anti-PD-1-treated patients, illustrating the exceptional benefit of Ipilimumab for immunotherapy, irAE remain a major threat to treated patients. It not only prevents many patients from continuing immunotherapy, but also limits the efficacy of CITE.

Recent studies provide a new perspective on these challenges. It has been demonstrated that cancer immunotherapeutic effects (CITE) and irAE represent distinct activities of anti-CTLA-4 antibodies. The former is due to selective depletion of regulatory T cells (Tregs) in the tumor microenvironment, while the latter is attributable to loss of cell-surface CTLA-4. Specifically, it has been shown that binding sensitivity of anti-CTLA-4 antibody to CTLA-4 at intracellular pH is key to controlling irAE. A pH-insensitive version of the antibody permits sustained binding to CTLA-4 after antibody-induced endocytosis, which triggers lysosomal degradation of CTLA-4, thereby causing irAE. By contrast, an antibody that has lower binding affinity at low pH disengages from CTLA-4 during trafficking through a low pH compartment, which enables CTLA-4 to recycle to the plasma membrane, thereby preventing irAE. More importantly, a pH-sensitive antibody that allows the recycling of CTLA-4 to the cell surface of Treg within the tumor microenvironment (TME) triggers stronger antibody-dependent cellular cytotoxicity/antibody-dependent cellular phagocytosis (ADCC/ADCP) due to higher target density, resulting in more efficient depletion of Treg, and better CITE in tumors. This new understanding makes it possible to refine CTLA-4 targeting and uncouple irAE from CITE by altering the protein sequence of the antibody to change its pH sensitivity. Ipilimumab is a pH-insensitive antibody which contributes to its propensity for irAE. Accordingly, there is a need in the art for pH-sensitive Ipilimumab variants with a reduced risk of causing irAE.

### SUMMARY OF THE INVENTION

Provided herein is an anti-CTLA antibody. The anti-CTLA antibody may comprise a light chain variable region and a heavy chain variable region. The light chain variable region may comprise a complementarity determining region (CDR) 1 comprising the sequence set forth in SEQ ID NO: 1, a CDR2 comprising the sequence set forth in SEQ ID NO: 3, and a CDR3 comprising the sequence set forth in SEQ ID NO: 5; and the heavy chain variable region may comprise a CDR1 comprising the sequence set forth in SEQ ID NO: 7, a CDR2 comprising the sequence set forth in SEQ ID NO: 8, and a CDR3 comprising the sequence set forth in SEQ ID NO: 12. The light chain variable region may comprise a CDR1 comprising the sequence set forth in SEQ ID NO: 2, a CDR2 comprising the sequence set forth in SEQ ID NO: 3, and a CDR3 comprising the sequence set forth in SEQ ID NO: 4; and the heavy chain may comprise a CDR1 comprising the sequence set forth in SEQ ID NO: 6, a CDR2 comprising the sequence set forth in SEQ ID NO: 10, and a CDR3 comprising the sequence set forth in SEQ ID NO: 12. The light chain variable region may comprise a CDR1 comprising the sequence set forth in SEQ ID NO: 2, a CDR2 comprising the sequence set forth in SEQ ID NO: 3, and a CDR3 comprising the sequence set forth in SEQ ID NO: 4; and the heavy chain variable region may comprise a CDR1 comprising the sequence set forth in SEQ ID NO: 6, a CDR2 comprising the sequence set forth in SEQ ID NO: 11, and a CDR3 comprising the sequence set forth in SEQ ID NO: 12.

The light chain variable region may comprise the sequence set forth in SEQ ID NO: 16 and the heavy chain variable region may comprise the sequence set froth in SEQ ID NO: 28. The light chain variable region may comprise the sequence set forth in SEQ ID NO: 15 and the heavy chain variable region may comprise the sequence set forth in SEQ ID NO: 26. The light chain variable region may comprise the sequence set forth in SEQ ID NO: 15 and the heavy chain variable region comprising the sequence set forth in SEQ ID NO: 27.

The anti-CTLA-4 antibody may comprise a light chain and a heavy chain. The light chain may comprise the sequence set forth in SEQ ID NO: 21 and the heavy chain may comprise the sequence set forth in SEQ ID NO: 35. The light chain may comprise the sequence set forth in SEQ ID NO: 20 and the heavy chain may comprise the sequence set forth in SEQ ID NO: 33. The light chain may comprise the sequence set forth in SEQ ID NO: 20 and the heavy chain may comprise the sequence set forth in SEQ ID NO: 34.

The anti-CTLA-4 antibody may bind to CTLA-4 with reduced affinity at a pH of about 5.5-6.0 as compared to Ipilimumab. The anti-CTLA-4 antibody may bind to CTLA-4 with reduced affinity at a pH of about 6.0 as compared to Ipilimumab.

Also provided herein is a composition comprising the anti-CTLA-4 antibody and a pharmaceutically acceptable excipient. The composition may comprise 20 mM histidine buffer, 8.8% (w/v) α, α-trehalose dihydrate, 0.06% (w/v) PS80, and 0.2 mM EDTA·2Na·2H₂O. The composition may have a pH of about 6.0. The composition may further comprise an anti-PD-1 antibody. The anti-PD-1 antibody may comprise a light chain comprising the sequence set forth in SEQ ID NO: 36 and a heavy chain comprising the sequence set forth in SEQ ID NO: 37.

Provided herein is a method of treating a cancer in a subject in need thereof, which may comprise administering the anti-CTLA-4 antibody or the composition. Also provided herein are the anti-CTLA-4 antibody or the composition for use in treating the cancer, and use of the anti-CTLA-4 antibody or the composition in the manufacture of a medicament for treating the cancer. The cancer may be melanoma, non-small cell lung carcinoma (NSCLC), HNSCC, ovarian cancer, endometrial carcinoma, cervical cancer, renal cell carcinoma, bladder cancer, esophageal cancer, gastric cancer, gastroesophageal (GE) junction cancer, colorectal cancer, anal cancer, hepatocellular carcinoma, cancer of a bile duct, adenoid cystic carcinoma (ACC), or triple negative breast cancer (TNBC). The cancer may be a solid tumor. The anti-CTLA-4 antibody or the composition may be administered or may be intended to be administered intravenously. Up to 10 mg/kg of the anti-CTLA-4 antibody may be administered or be intended to be administered to the subject. The administration may be or may be intended to be once every 3 weeks.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the design of pH-sensitive Ipilimumab variants in which complementarity determining regions (CDRs) include histidine substitutions of tyrosine residues. The 23 variants include different combinations of Y to H substitutions among the CDRs. LH00 (light chain VLM0 and heavy chain LHM0): wild-type Ipilimumab. Light chain variable regions VLM0-VLM3 have SEQ ID NOs: 14-17, respectively, and heavy chain variable regions VHM0-VHM5 have SEQ ID NOs: 23-28, respectively.

FIGS. 2A-D show analyses of pH-sensitive Ipilimumab variants generated by introducing histidine to tyrosine substitutions in complementarity determining regions (CDRs). His-hCTLA-4 (0.5 g/ml) was coated in ELISA plates and different anti-CTLA4-mAbs were added at 1 μg/ml in the buffer at different pH range from pH 4.5 to 7.0. Antibodies bound to CTLA-4 were measured using horseradish peroxidase-labeled anti-human IgG antibodies. The pH binding of CTLA-4 between Ipilimumab variants and wild-type antibodies are shown in different combination of mutations. In each of FIGS. 2A-D, the Ipilimumab variants include various combinations of Y to H substitutions in at least one of the heavy and light chain variable region. In each of these, VHM0-VHM5 have SEQ ID NOs: 23-28, respectively. FIG. 2A shows mutants with only substitutions in the heavy chain variable region. VLM0 has SEQ ID NO: 14. FIG. 2B shows mutants with Y to H substitutions in both Ys in the light chain variable region. VLM3 has SEQ ID NO: 17. FIG. 2C shows mutants with only a Y to H substitution in the CDR1 of the light chain. VLM1 has SEQ ID NO: 15. FIG. 2D shows mutants with only a Y to H substitution in the CDR3 of the light chain variable region. VLM2 has SEQ ID NO: 16.

FIGS. 3A-B show the pH binding sensitivity of Ipilimumab variants LH13, LH14, and LH25 compared with wild-type (LH00) and pH-sensitive antibody HL12. The methods were performed as described in FIG. 2. FIG. 3A shows raw data. FIG. 3B shows data normalized to pH 7. LH13: (Light chain Y33H; Heavy chain Y59H); LH14: (Light chain Y33H; Heavy chain Y58H); LH25: (Light chain Y92H; Heavy chain Y32H).

FIG. 4 shows that a pH-6-sensitive Ipilimumab variant, but not a pH-6-insensitive variant, dissociated from CTLA-4 during antibody-induced internalization. 293T stable cell lines expressing hCTLA-4 were labeled with anti-CTLA-4 mAbs at 4° C. for 30 min. After washing out unbound antibodies, cells were transferred to 37° C. for Ih. Antibody-bound surface CTLA-4 was captured by protein-G beads and tested by Western blot.

FIG. 5 shows surface plasmon resonance (SPR) measurements of the affinities of Ipilimumab variants at different pH. Ipilimumab, LH13, and LH25 were assessed for their binding affinity to his-CTLA-4 by SPR analysis using a Biacore T100 biosensor (GE Healthcare). 1000 response units (RU) of Protein A from Staphylococcus aureus (Sigma-Aldrich) were immobilized on flow cells 1 and 2 of a Series S Sensor Chip CM5 (GE, BR100530). Approximately 150 RUs of antibody were directly captured on flow cell 2. Binding experiments were carried out in 10 mM HEPES, 150 mM NaCl, 0.05% (v/v) Tween 20 with pH 7.4 or pH 6 at 25° C. A two-fold titration series of his-CTLA-4 (0.46875 nM-60 nM for pH 7.4/0.976 nM-60 nM for pH 6) were used. In between runs, the sensor surface was regenerated with two 45 sec injections of 20 mM HCl. Sensorgrams were double referenced against the control flow cell (Fc1) and three buffer injections.

FIG. 6 shows the binding effect of Ipilimumab and pH-sensitive variants to cell surface CTLA-4. 293T stable cell lines expressing hCTLA-4 were treated with Ipilimumab and its variants, LH13 and LH25 at 37° C. for 4 hr, and then plasma membrane proteins of treated cells were isolated by using a MINUTE™ plasma membrane protein isolation and cell fractionation kit (SM-005, Invent Biotechnologies, INC.). Cell surface CTLA-4 was tested by Western blotting using CTLA-4 Antibody (H-126, sc-9094). Tubulin and Na⁺/K⁺ ATPase alpha1 were used as the cytosolic fraction marker and the plasma membrane marker, separately.

FIG. 7 shows that a pH-6-insensitive Ipilimumab variant, but not a pH-6-sensitive variant, goes to the lysosome after internalization. Ipilimumab WT, LH13, or LH25 was labeled with AF488 and treated with CHO stable cell lines expressing hCTLA-4 at 4° C. After excess antibodies were washed away, cells were incubated at 37° C. for 30 min and further stained with lysotracker. Co-localization between AF488-labeled anti-CTLA-4 mAbs and lysosomes was shown by confocal images (green: anti-CTLA-4 mAbs; magenta: Lysosomes; white: overlap of green and magenta). Scale bar: 10 m.

FIG. 8 shows that a pH-6-sensitive Ipilimumab variant, but not a pH-6-insensitive variant, rescues CTLA-4 from lysosomal degradation. Ipilimumab WT, LH13, or LH25 was labeled with AF488 and treated with CHO stable cell lines expressing hCTLA-4-OPF at 4° C. After extra antibodies were washed away, cells were incubated at 37° C. for 30 min and further stained with lysotracker. Co-localization of AF488-labeled anti-CTLA-4 mAbs, lysosomes and orange-fluorescence protein (OFP)-tagged CTLA-4 was shown by representative confocal images (Green: anti-CTLA-4 mAbs; red: CTLA-4; blue: Lysosomes; white: overlapping of the three markers). Scale bar: 10 m.

FIGS. 9A-B show that a pH-6-sensitive Ipilimumab variant rescues combination therapy-induced body weight loss and anemia. FIG. 9A. C57BL/6 Ctla4 h/h mice were treated, respectively, with control human IgG-Fc+anti-PD1, Ipilimumab+anti-PD1, LH13+anti-PD1, and LH25+anti-PD1 at a dose of 100 μg/mouse/injection on days 10, 13, 16 and 19. Major growth retardation was measured by every three days. One mouse from the human IgG-Fc+anti-PD-1 and two mice from the Ipilimumab plus anti-PD-1 and LH13+anti-PD-1-treated groups were excluded from analysis due to death before the endpoint of the experiments with serious grow retardation. FIG. 9B. CBC analysis was performed on day 39 after birth. To avoid cage variation, mice in the same cages were individually tagged and treated with different antibodies. Data are mean±S.E.M., n=22-24 mice per group. Tests were performed double blind. The samples were collected from three independent experiments. Two-way repeat measurement ANOVA with Bonferroni multiple comparison test was used for statistical tests. *** P<0.001; **** P<0.0001.

FIGS. 10A-B show that a pH-6-sensitive Ipilimumab variant rescues combination therapy-induced multiple organ inflammation. The same mice in the experiments of FIG. 9A-B were analyzed. The necropsy was performed on day 40 afterbirth. FIG. 10A. Representative images of H&E-stained paraffin sections from different organs. Representative inflammatory foci are marked with arrows. Scale bar, 200 m. FIG. 10B. Toxicity scores of internal organs and glands in a. The samples were collected from three independent experiments and have been scored double blind. Data were analyzed by one-way ANOVA with Bonferroni's multiple comparison test. **** P<0.0001.

FIGS. 11A-B show the immunotherapeutic effects of Ipilimumab and its pH-sensitive variants. FIG. 11A shows tumor growth in mice receiving either hIgG-Fc, Ipilimumab, LH13 or LH25 in YM3.3 melanoma tumors (left panel) or MC38 colon tumors (right panel). In the YM3.3 melanoma model, mice (n=13-14) were treated with 3 doses of 1.5 mg/kg/dose of antibodies on day 10, 13 and day 16 after tumor inoculation. The same treatments were conducted in the MC38 colon cancer model (n=12-13) with 2 doses of antibodies on day 17 and day 20 after tumor inoculation. FIG. 11B shows Kaplan-Meier survival curves of tumor-bearing mice in FIG. 11A. Data are mean±S.E.M., and from two independent experiments. Two-way repeat measurement ANOVA with Bonferroni multiple comparison test was used for statistical tests. * P<0.05; ** P<0.01; *** P<0.001; **** P<0.0001.

FIG. 12 shows Treg depletion of Ipilimumab and its pH-sensitive variants in the tumor microenvironment and spleen. YM3.3-bearing Ctla4^{h/h} mice (n=7) were treated with either control hIgG, Ipilimumab, LH13 or LH25 (100 g/mouse) on day 14 after tumor inoculation. Treg depletion in the tumor microenvironment (left panel) and in spleen (right panel) was determined by the percentage of Treg cells among CD4 T cells at 16 h after antibody treatment. Data are mean±S.E.M., and from two independent experiments. Two-way repeat measurement ANOVA with Bonferroni multiple comparison test was used for statistical tests. * P<0.05; ** P<0.01.

### DETAILED DESCRIPTION

The inventors have discovered pH-sensitive Ipilimumab variants that surprisingly abrogate irAE while improving on their CITE.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, the term “antibody” refers to an immunoglobulin molecule that possesses a “variable region” antigen recognition site. The term “variable region” refers to a domain of the immunoglobulin that is distinct from a domain broadly shared by antibodies (such as an antibody Fc domain). The variable region comprises a “hypervariable region” whose residues are responsible for antigen binding. The hypervariable region comprises amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; ref 44) and may comprise those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain). “Framework Region” or “FR” residues are those variable domain residues other than the hypervariable region residues as herein defined. An antibody disclosed herein may be a monoclonal antibody, multi-specific antibody, human antibody, humanized antibody, synthetic antibody, chimeric antibody, camelized antibody, single chain antibody, disulfide-linked Fv (sdFv), intrabody, or an anti-idiotypic (anti-Id) antibody (including, e.g., anti-Id and anti-anti-Id antibodies to antibodies of the invention). In particular, the antibody may be an immunoglobulin molecule, such as IgG, IgE, IgM, IgD, IgA or IgY, or be of a class, such as IgG₁, IgG₂, IgG₃, IgG₄, IgA₁ or IgA₂, or of a subclass.

As used herein, the term “antigen binding fragment” of an antibody refers to one or more portions of an antibody that contain the antibody's Complementarity Determining Regions (“CDRs”) and optionally the framework residues that comprise the antibody's “variable region” antigen recognition site, and exhibit an ability to immunospecifically bind antigen. Such fragments include Fab′, F(ab′)₂, Fv, single chain (ScFv), and mutants thereof, naturally occurring variants, and fusion proteins comprising the antibody's “variable region” antigen recognition site and a heterologous protein (e.g., a toxin, an antigen recognition site for a different antigen, an enzyme, a receptor or receptor ligand, etc.). As used herein, the term “fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues.

Human, chimeric or humanized antibodies are particularly preferred for in vivo use in humans, however, murine antibodies or antibodies of other species may be advantageously employed for many uses (for example, in vitro or in situ detection assays, acute in vivo use, etc.).

A “chimeric antibody” is a molecule in which different portions of the antibody are derived from different immunoglobulin molecules such as antibodies having a variable region derived from a non-human antibody and a human immunoglobulin constant region. Chimeric antibodies comprising one or more CDRs from a non-human species and framework regions from a human immunoglobulin molecule can be produced using a variety of techniques known in the art including, for example, CDR-grafting (EP 239,400; International Publication No. WO 91/09967; and U.S. Pat. Nos. 5,225,539, 5,530,101, and 5,585,089), veneering or resurfacing (EP 592,106; EP 519,596; 46-48), and chain shuffling (U.S. Pat. No. 5,565,332), the contents of all of which are incorporated herein by reference.

Antibodies described herein may be humanized antibodies. As used herein, the term “humanized antibody” refers to an immunoglobulin comprising a human framework region and one or more CDRs from a non-human (usually a mouse or rat) immunoglobulin. The non-human immunoglobulin providing the CDRs is called the “donor” and the human immunoglobulin providing the framework is called the “acceptor.” Constant regions need not be present, but if they are, they must be substantially identical to human immunoglobulin constant regions, i.e., at least about 85-90%, preferably about 95% or more identical. Hence, all parts of a humanized immunoglobulin, except possibly the CDRs, are substantially identical to corresponding parts of natural human immunoglobulin sequences. A humanized antibody is an antibody comprising a humanized light chain and a humanized heavy chain immunoglobulin. For example, a humanized antibody would not encompass a typical chimeric antibody, because, e.g., the entire variable region of a chimeric antibody is non-human. The donor antibody is referred to as being “humanized,” by the process of “humanization,” because the resultant humanized antibody is expected to bind to the same antigen as the donor antibody that provides the CDRs. Humanized antibodies may be human immunoglobulins (recipient antibody) in which hypervariable region residues of the recipient are replaced by hypervariable region residues from a non-human species (donor antibody) such as mouse, rat, rabbit or a non-human primate having the desired specificity, affinity, and capacity. In some instances, Framework Region (FR) residues of the human immunoglobulin are replaced by corresponding non-human residues. Furthermore, humanized antibodies may comprise residues which are not found in the recipient antibody or in the donor antibody. These modifications may further refine antibody performance. The humanized antibody may comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody may optionally also comprise at least a portion of an immunoglobulin constant region (Fc), which may be that of a human immunoglobulin that immunospecifically binds to an FcγRIIB polypeptide, that has been altered by the introduction of one or more amino acid residue substitutions, deletions, or additions (i.e., mutations).

### 2. pH-Sensitive Ipilimumab Variants

Provided herein are anti-CTLA-4 antibodies and antigen-binding fragments thereof. The anti-CTLA-4 antibody may immunospecifically bind to CTLA-4, particularly human CTLA-4. The CTLA-4 may be expressed on the surface of a live cell at an endogenous or transfected concentration. The live cell may be a T cell, which may be a regulatory T cell (Treg). The antibody may be a monoclonal antibody, a human antibody, a chimeric antibody or a humanized antibody. The antibody may also be monospecific, bispecific, trispecific, or multispecific. The antibody may be detectably labeled, and may comprise a conjugated toxin, drug, receptor, enzyme, or receptor ligand. The anti-CTLA-4 antibody may induce cancer rejection while also reducing immune-related adverse effects (irAE) associated with immunotherapy. The anti-CTLA-4 antibody may efficiently induce Fc receptor-dependent Treg depletion and tumor rejection. The anti-CTLA-4 antibody may dissociate from CTLA-4 under intracellular acidic pH conditions, which may allow CTLA-4 to recycle back to the cell surface.

The anti-CTLA-4 antibody may be a variant of Ipilimumab. Compared to Ipilimumab, the anti-CTLA-4 antibody may be pH-sensitive, and may exhibit reduced binding to CTLA-4 at an acidic pH. The pH at which the pH-sensitive anti-CTLA-4 antibody exhibits reduced binding to CTLA-4 may be about 4.5, 5.0, 5.5, 6.0, or 6.5. The pH may be 4.5-6.0, and in particular may be about 5.5 or 6.0. In one example, the pH at which the anti-CTLA-4 antibody exhibits reduced binding affinity for CTLA-4 may be about 6.0.

In comparison to the wild-type Ipilimumab, the amino acid sequence of the variant may comprise one or more substitutions of tyrosine with histidine near or within one or more complementarity determining region (CDR) of at least one heavy or light chain. The CDR may be one or more of CDR1, CDR2, and CDR3. The anti-CTLA-4 antibody may comprise a light chain variable region, which may comprise one or more of the following CDRs.

TABLE 1
Light chain variable region CDR sequences

| CDR1 | CDR2 | CDR3 |
|---|---|---|
| RASQSVGSSYLA | GAFSRAT | QQYGSSPWT |
| (SEQ ID NO: 1) | (SEQ ID NO: 3) | (SEQ ID NO: 4) |
| RASQSVGSSHLA | | QQHGSSPWT |
| (SEQ ID NO: 2) | | (SEQ ID NO: 5) |

The light chain variable region may comprise a CDR1, CDR2, and CDR3 respectively comprising the sequences set forth in the table below.

TABLE 2
Light chain variable region CDR combinations

| Light | | | |
|---|---|---|---|
| chain | CDR1 SEQ ID NO: | CDR2 SEQ ID NO: | CDR3 SEQ ID NO: |
| VLM0 | 1 | 3 | 4 |
| VLM1 | 2 | 3 | 4 |
| VLM2 | 1 | 3 | 5 |
| VLM3 | 2 | 3 | 5 |

The anti-CTLA-4 antibody may comprise a heavy chain variable region, which may comprise one or more of the following CDRs.

TABLE 3
Heavy chain variable region CDR sequences

| CDR1 | CDR2 | CDR3 |
|---|---|---|
| GETFSSYTMH | ISYDGNNKYYADSVKG | TGWLGPEDY |
| (SEQ ID NO: 6) | (SEQ ID NO: 8) | (SEQ ID NO: 12) |
| GFTFSSHTMH | ISYDGNNKHHADSVKG | TGWLGPFDH |
| (SEQ ID NO: 7) | (SEQ ID NO: 9) | (SEQ ID NO: 13) |
| | ISYDGNNKHYADSVKG | |
| | (SEQ ID NO: 10) | |
| | ISYDGNNKYHADSVKG | |
| | (SEQ ID NO: 11) | |

The heavy chain variable region may comprise a CDR1, CDR2, and CDR3 respectively comprising the sequences set forth in the table below.

TABLE 4
Heavy chain variable region CDR combinations

| Heavy | | | |
|---|---|---|---|
| chain | CDR1 SEQ ID NO: | CDR2 SEQ ID NO: | CDR3 SEQ ID NO: |
| VHM0 | 6 | 8 | 12 |
| VHM1 | 7 | 9 | 13 |
| VHM2 | 6 | 8 | 13 |
| VHM3 | 6 | 10 | 12 |
| VHM4 | 6 | 11 | 12 |
| VHM5 | 7 | 8 | 12 |

The light chain variable region may comprise one of the following sequences.

VLM0
(SEQ ID NO: 14)
EIVLTQSPGTLSLSPGERATLSCRASQSVGSSYLAWYQQKPGQAPRLLIY
GAFSRATGIPDRFSGSGSGTDFTLTISRLEPEDFAVYYCQQYGSSPWTFG
QGTKVEIK
VLM1
(SEQ ID NO: 15)
EIVLTQSPGTLSLSPGERATLSCRASQSVGSSHLAWYQQKPGQAPRLLIY
GAFSRATGIPDRFSGSGSGTDFTLTISRLEPEDFAVYYCQQYGSSPWTFG
QGTKVEIK
VLM2
(SEQ ID NO: 16)
EIVLTQSPGTLSLSPGERATLSCRASQSVGSSYLAWYQQKPGQAPRLLIY
GAFSRATGIPDRFSGSGSGTDFTLTISRLEPEDFAVYYCQQHGSSPWTFG
QGTKVEIK
VLM3
(SEQ ID NO: 17)
EIVLTQSPGTLSLSPGERATLSCRASQSVGSSHLAWYQQKPGQAPRLLIY
GAFSRATGIPDRFSGSGSGTDFTLTISRLEPEDFAVYYCQQHGSSPWTFG
QGTKVEIK

The light chain may comprise a constant region comprising the following sequence.

(SEQ ID NO: 18)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

The light chain may comprise one of the following sequences.

VLM0
(SEQ ID NO: 19)
EIVLTQSPGTLSLSPGERATLSCRASQSVGSSYLAWYQQKPGQAPRLLIY
GAFSRATGIPDRFSGSGSGTDFTLTISRLEPEDFAVYYCQQYGSSPWTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
VLM1
(SEQ ID NO: 20)
EIVLTQSPGTLSLSPGERATLSCRASQSVGSSHLAWYQQKPGQAPRLLIY
GAFSRATGIPDRFSGSGSGTDFTLTISRLEPEDFAVYYCQQYGSSPWTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
VLM2
(SEQ ID NO: 21)
EIVLTQSPGTLSLSPGERATLSCRASQSVGSSYLAWYQQKPGQAPRLLIY
GAFSRATGIPDRFSGSGSGTDFTLTISRLEPEDFAVYYCQQHGSSPWTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
VLM3
(SEQ ID NO: 22)
EIVLTQSPGTLSLSPGERATLSCRASQSVGSSHLAWYQQKPGQAPRLLIY
GAFSRATGIPDRFSGSGSGTDFTLTISRLEPEDFAVYYCQQHGSSPWTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC

The heavy chain variable region may comprise one of the following sequences.

VHM0
(SEQ ID NO: 23)
QVQLVESGGGVVQPGRSLRLSCAASGFTFSSYTMHWVRQAPGKGLEWVTF
ISYDGNNKYYADSVKGRFTISRDNSKNTLYLQMNSLRAEDTAIYYCARTG
WLGPFDYWGQGTLVTVSS
VHM1
(SEQ ID NO: 24)
QVQLVESGGGVVQPGRSLRLSCAASGFTFSSHTMHWVRQAPGKGLEWVTF
ISYDGNNKHHADSVKGRFTISRDNSKNTLYLQMNSLRAEDTAIYYCARTG
WLGPFDHWGQGTLVTVSS
VHM2
(SEQ ID NO: 25)
QVQLVESGGGVVQPGRSLRLSCAASGFTFSSYTMHWVRQAPGKGLEWVTF
ISYDGNNKYYADSVKGRFTISRDNSKNTLYLQMNSLRAEDTAIYYCARTG
WLGPFDHWGQGTLVTVSS
VHM3
(SEQ ID NO: 26)
QVQLVESGGGVVQPGRSLRLSCAASGFTFSSYTMHWVRQAPGKGLEWVTF
ISYDGNNKHYADSVKGRFTISRDNSKNTLYLQMNSLRAEDTAIYYCARTG
WLGPFDYWGQGTLVTVSS
VHM4
(SEQ ID NO: 27)
QVQLVESGGGVVQPGRSLRLSCAASGFTFSSYTMHWVRQAPGKGLEWVTF
ISYDGNNKYHADSVKGRFTISRDNSKNTLYLQMNSLRAEDTAIYYCARTG
WLGPFDYWGQGTLVTVSS
VHM5
(SEQ ID NO: 28)
QVQLVESGGGVVQPGRSLRLSCAASGFTFSSHTMHWVRQAPGKGLEWVTF
ISYDGNNKYYADSVKGRFTISRDNSKNTLYLQMNSLRAEDTAIYYCARTG
WLGPFDYWGQGTLVTVSS

The anti-CTLA-4 antibody may comprise a heavy chain constant region from a human Ig protein, which may be IgG, IgE, IgM, IgD, IgA, IgY, IgG1, IgG2, IgG3, IgG4, IgA₁ or IgA₂. In one example, the constant region is a Fc region from a human IgG1 protein. The heavy chain may comprise a constant region comprising the following sequence.

(SEQ ID NO: 29)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

The heavy chain constant region may comprise the following sequence.

(SEQ ID NO: 40)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG

The heavy chain constant region may comprise the following sequence.

(SEQ ID NO: 38)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG

A C-terminal lysine (K) may be additionally included in the amino acid sequence of the heavy chain set forth in SEQ ID NO: 38, which may increase expression levels. The terminal lysine may be cleaved naturally during production of the anti-CTLA-4 antibody, or upon administration of the antibody.

The heavy chain constant region may also comprise one or more mutations. Relative to the sequence set forth in SEQ ID NO: 29, 38 or 40, the one or more mutations may be selected from M135Y, S137T, T139E, S181A, E216A, and K217A, and a combination thereof. In one example, the heavy chain constant region of the antibody comprises all six mutations. The mutant heavy chain constant region may comprise the following sequence.

(SEQ ID NO: 39)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLYITREPEVTCVVVDVS
HEDPEVKFNWYVDGVEVHNAKTKPREEQYNATYRVVSVLTVLHQDWLNGK
EYKCKVSNKALPAPIAATISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG

The heavy chain may comprise one of the following sequences.

VHM0
(SEQ ID NO: 30)
QVQLVESGGGVVQPGRSLRLSCAASGFTFSSYTMHWVRQAPGKGLEWVTF
ISYDGNNKYYADSVKGRFTISRDNSKNTLYLQMNSLRAEDTAIYYCARTG
WLGPFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
VHM1
(SEQ ID NO: 31)
QVQLVESGGGVVQPGRSLRLSCAASGFTFSSHTMHWVRQAPGKGLEWVTF
ISYDGNNKHHADSVKGRFTISRDNSKNTLYLQMNSLRAEDTAIYYCARTG
WLGPFDHWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
VHM2
(SEQ ID NO: 32)
QVQLVESGGGVVQPGRSLRLSCAASGFTFSSYTMHWVRQAPGKGLEWVTF
ISYDGNNKYYADSVKGRFTISRDNSKNTLYLQMNSLRAEDTAIYYCARTG
WLGPFDHWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
VHM3
(SEQ ID NO: 33)
QVQLVESGGGVVQPGRSLRLSCAASGFTFSSYTMHWVRQAPGKGLEWVTF
ISYDGNNKHYADSVKGRFTISRDNSKNTLYLQMNSLRAEDTAIYYCARTG
WLGPFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
VHM4
(SEQ ID NO: 34)
QVQLVESGGGVVQPGRSLRLSCAASGFTFSSYTMHWVRQAPGKGLEWVTF
ISYDGNNKYHADSVKGRFTISRDNSKNTLYLQMNSLRAEDTAIYYCARTG
WLGPFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
VHM5
(SEQ ID NO: 35)
QVQLVESGGGVVQPGRSLRLSCAASGFTFSSHTMHWVRQAPGKGLEWVTF
ISYDGNNKYYADSVKGRFTISRDNSKNTLYLQMNSLRAEDTAIYYCARTG
WLGPFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

The anti-CTLA-4 antibody may comprise a combination of a light chain variable region having a combination of CDR1-3 of Table 2 and a heavy chain variable region having a combination of CDR1-3 of Table 4. In one example, the light chain variable region is VLM0 and the heavy chain variable region is VHM0, referred to herein as LH00. In another example the light chain variable region is VLM0 and the heavy chain variable region is VHM1, referred to herein as LH01. The anti-CTLA-4 antibody may comprise a combination of a light chain variable region and a heavy chain variable region selected from LH01, LH02, LH03, LH04, LH05, LH10, LH11, LH12, LH13, LH14, LH15, LH20, LH21, LH22, LH23, LH24, LH25, LH30, LH31, LH32, LH33, LH34, and LH35. The anti-CTLA-4 antibody may comprise one of the following combinations of light chain variable region and heavy chain variable region.

TABLE 5

| | Light chain variable region | Heavy chain variable region |
|---|---|---|
| Combination | (SEQ ID NO:) | (SEQ ID NO:) |
| LH00 | 14 | 23 |
| LH01 | 14 | 24 |
| LH02 | 14 | 25 |
| LH03 | 14 | 26 |
| LH04 | 14 | 27 |
| LH05 | 14 | 28 |
| LH10 | 15 | 23 |
| LH11 | 15 | 24 |
| LH12 | 15 | 25 |
| LH13 | 15 | 26 |
| LH14 | 15 | 27 |
| LH15 | 15 | 28 |
| LH20 | 16 | 23 |
| LH21 | 16 | 24 |
| LH22 | 16 | 25 |
| LH23 | 16 | 26 |
| LH24 | 16 | 27 |
| LH25 | 16 | 28 |
| LH30 | 17 | 23 |
| LH31 | 17 | 24 |
| LH32 | 17 | 25 |
| LH33 | 17 | 26 |
| LH34 | 17 | 27 |
| LH35 | 17 | 28 |

The anti-CTLA-4 antibody may comprise a combination of a light chain and a heavy chain selected from LH01, LH02, LH03, LH04, LH05, LH10, LH11, LH12, LH13, LH14, LH15, LH20, LH21, LH22, LH23, LH24, LH25, LH30, LH31, LH32, LH33, LH34, and LH35. The anti-CTLA-4 antibody may comprise one of the following combinations of light chain and heavy chain.

TABLE 6
Full structures of Ipilimumab and variants thereof

| | Light chain | Heavy chain |
|---|---|---|
| Combination | (SEQ ID NO:) | (SEQ ID NO:) |
| LH00 | 19 | 30 |
| LH01 | 19 | 31 |
| LH02 | 19 | 32 |
| LH03 | 19 | 33 |
| LH04 | 19 | 34 |
| LH05 | 19 | 35 |
| LH10 | 20 | 30 |
| LH11 | 20 | 31 |
| LH12 | 20 | 32 |
| LH13 | 20 | 33 |
| LH14 | 20 | 34 |
| LH15 | 20 | 35 |
| LH20 | 21 | 30 |
| LH21 | 21 | 31 |
| LH22 | 21 | 32 |
| LH23 | 21 | 33 |
| LH24 | 21 | 34 |
| LH25 | 21 | 35 |
| LH30 | 22 | 30 |
| LH31 | 22 | 31 |
| LH32 | 22 | 32 |
| LH33 | 22 | 33 |
| LH34 | 22 | 34 |
| LH35 | 22 | 35 |

In one example, the anti-CTLA-4 antibody exhibits reduced binding to CTLA-4 at about pH 5.5, and the anti-CTLA-4 antibody may be LH13, LH14, or LH25. In another example, the anti-CTLA-4 antibody exhibits reduced binding to CTLA-4 at about pH 6.0, and the anti-CTLA-4 antibody may be LH25.

LH25 comprises a light chain variable region comprising a CDR1 comprising the sequence set forth in SEQ ID NO: 1, a CDR2 comprising the sequence set forth in SEQ ID NO: 3, and a CDR3 comprising the sequence set forth in SEQ ID NO: 5. LH25 comprises a heavy chain variable region comprising a CDR1 comprising the sequence set forth in SEQ ID NO: 7, a CDR2 comprising the sequence set forth in SEQ ID NO: 8, and a CDR3 comprising the sequence set forth in SEQ ID NO: 12. LH25 comprises a light chain variable region comprising the sequence set forth in SEQ ID NO: 16 and a heavy chain variable region comprising the sequence set forth in SEQ ID NO: 28. LH25 comprises a light chain comprising the sequence set forth in SEQ ID N: 21 and a heavy chain comprising the sequence set forth in SEQ ID NO: 35.

LH13 comprises a light chain variable region comprising a CDR1 comprising the sequence set forth in SEQ ID NO: 2, a CDR2 comprising the sequence set forth in SEQ ID NO: 3, and a CDR3 comprising the sequence set forth in SEQ ID NO: 4. LH13 comprises a heavy chain variable region comprising a CDR1 comprising the sequence set forth in SEQ ID NO: 6, a CDR2 comprising the sequence set forth in SEQ ID NO: 10, and a CDR3 comprising the sequence set forth in SEQ ID NO: 12. LH13 comprises a light chain variable region comprising the sequence set forth in SEQ ID NO: 15 and a heavy chain variable region comprising the sequence set forth in SEQ ID NO: 26. LH13 comprises a light chain comprising the sequence set forth in SEQ ID N: 20 and a heavy chain comprising the sequence set forth in SEQ ID NO: 33.

LH14 comprises a light chain variable region comprising a CDR1 comprising the sequence set forth in SEQ ID NO: 2, a CDR2 comprising the sequence set forth in SEQ ID NO: 3, and a CDR3 comprising the sequence set forth in SEQ ID NO: 4. LH14 comprises a heavy chain variable region comprising a CDR1 comprising the sequence set forth in SEQ ID NO: 6, a CDR2 comprising the sequence set forth in SEQ ID NO: 11, and a CDR3 comprising the sequence set forth in SEQ ID NO: 12. LH14 comprises a light chain variable region comprising the sequence set forth in SEQ ID NO: 15 and a heavy chain variable region comprising the sequence set forth in SEQ ID NO: 27. LH14 comprises a light chain comprising the sequence set forth in SEQ ID N: 20 and a heavy chain comprising the sequence set forth in SEQ ID NO: 34.

### 3. Pharmaceutical Compositions

Provided herein is a composition comprising the anti-CTLA-4 antibody. The composition may comprise a pharmaceutically acceptable excipient. The composition may comprise a sterile solution for intravenous infusion. The composition may be a preservative-free aqueous solution, and may be suitable for parenteral administration. In one example, the composition independently comprises about 5, 10, 20, 30, 40, or 50 mg/mL, or an amount in a range of two of these amounts, of the anti-CTLA-4 antibody. The composition may comprise about 5, 10, or 20 mg/mL the anti-CTLA-4 antibody. In one example, the composition comprises 10 mg/mL of the anti-CTLA-4 antibody. In one example, the composition comprises 100 mg of the anti-CTLA-4 antibody. The composition may comprise about 5, 10, 15, 20, 25, or 30 mL of an aqueous solution of the antibody. The aqueous solution may be contained in a vial. The volume of the aqueous solution in the vial may be 10 mL. In one example the vial is filled with an extractable volume of 10 mL for a total of approximately 100 mg/vial of the anti-CTLA-4 antibody.

The pharmaceutically acceptable carrier may comprise one or more of a histidine buffer, an acetate buffer, trehalose, PS80, sucrose, and EDTA. The composition may comprise about 5, 10, 15, 20, 25, 30, 35, or 40 mM histidine buffer, or an amount in a range of two of these amounts. In one example, the composition comprises 20 mM histidine buffer. The composition may also comprise about 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0% (w/v) α, α-trehalose dihydrate, or an amount in a range of two of these amounts. In one example, the composition comprises 8.8% (w/v) α, α-trehalose dihydrate. The composition may comprise about 0.01, 0.02, 0.03, 0.04, 0.05, 0.06, 0.07, 0.08, 0.09, or 0.10 (w/v) PS80, or an amount in a range of two of these amounts. In one example, the composition comprises 0.06% (w/v) PS80. The composition may comprise about 0.05, 0.10, 0.15, 0.20, 0.25, 0.30, 0.35, or 0.40 mM EDTA, which may be EDTA·2Na·2H₂O. In one example, the composition comprises 0.2 mM EDTA. The composition may have a pH of 5.5, 5.6, 5.7, 5.8, 5.9, 6.0, 6.1, 6.2, 6.3, 6.4, or 6.5, or a pH in a range thereof.

The anti-CTLA-4 antibody may be co-administered, and may be co-formulated, with a second cancer therapy. The second cancer therapy may be an anti-PD-1 antibody, which may be pembrolizumab (KEYTRUDA), Nivolumab (OPDIVO)), or one described in U.S. Pat. No. 11,345,754, the contents of which are incorporated herein by reference. The anti-PD-1 antibody may comprise a light chain comprising the sequence set forth in SEQ ID NO: 36 and a heavy chain comprising the sequence set forth in SEQ ID NO: 37, as described below.

Light chain
(SEQ ID NO: 36)
DIQLTQSPSFLSASVGDRVTITCKASQDAGSAVAWYQQKPGKAPKLLIYW
ASTRHTGVPSRFSGSGSGTEFTLTISSLQPEDFATYYCQQYSSYPWTFGG
GTKLEIKRTVAAPSVFIFPPSDEQLKSGTASVVCLLNNFYPREAKVQWKV
DNALQSGNSQESVTEQDSKDSTYSLSSTLTKADYEKHKVYACEVTHQGLS
SPVTKSFNRGEC
Heavy chain
(SEQ ID NO: 37)
EVQLVESGGGLVQPGGSLRLSCAASGFTFSRYDMSWVRQAPGKGLEWVST
ISGGGRYTYYPDSVKGRFTISRDNSKNTLYLQMNSLRAEDTAVYYCTSPY
GNYGMDYWGQGTSVTVSSASTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFLGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGK

In one example, the composition independently comprises about 5, 10, 20, 30, 40, or 50 mg/mL, or an amount in a range of two of these amounts, of each of the anti-CTLA-4 antibody and the anti-PD-1 antibody. The composition may comprise about 5, 10, or 20 mg/mL of each of the anti-CTLA-4 antibody and the anti-PD-1 antibody. The composition may comprise an anti-CTLA-4 antibody-to-anti-PD-1 antibody ratio of about 10:1, 5:1, 4:1, 3:1, 2:1, 4:3, 3:2, 1:1, 2:3, 3:4, 1:2, 1:3, 1:4, 1:5, or 1:10. In one example, the composition comprises 10 mg/mL of each of the anti-CTLA-4 antibody and the anti-PD-1 antibody. In one example, the composition comprises 100 mg of each of the anti-CTLA-4 antibody and the anti-PD-1 antibody. The composition may comprise about 5, 10, 15, 20, 25, or 30 mL of an aqueous solution of the antibodies. The aqueous solution may be contained in a vial. The volume of the aqueous solution in the vial may be 10 mL. In one example, the anti-CTLA-4 and anti-PD-1 antibodies are present at a combined protein concentration of 20 mg/mL. Each vial may be filled with an extractable volume of 10 mL for a total of approximately 100 mg/vial of the anti-CTLA-4 antibody and 100 mg/vial of the anti-PD-1 antibody, respectively.

### 4. Dosing Regimens

The anti-CTLA-4 antibody may be administered systemically, which may be via injection or intravenous (IV) administration. One or more doses of the anti-CTLA-4 antibody may be administered to or be intended for administration to a subject. Independently, each dose of the anti-CTLA-4 antibody may be about 0.1 mg/kg, 0.3 mg/kg, 1 mg/kg, 3 mg/kg, 5, mg/kg, 6 mg/kg, 10 mg/kg, 15 mg/kg, or 20 mg/kg, or an amount in a range of two of these amounts. Each dose of the anti-CTLA-4 antibody may be about 50, 100, 150, 200, 250, 300, 350, 400, 450, 500, 550, 600, 650, 700, 750, 800, 850, 900, 950, 1000 mg, or an amount in a range of two of these amounts, for a subject with a body weight of 30 kg or higher. Each dose of the antibody anti-CTLA-4 may be about 50-1000 mg for a subject with a body weight of 30 kg or higher.

When the anti-CTLA-4 antibody is at a dose of 100 mg, then it may be infused or intended to be infused over a period of at least 30 min. When the anti-CTLA-4 antibody is at a dose of 200 mg or more, then it may be infused or intended to be infused over a period of at least 60 min.

The dose of the anti-CTLA-4 antibody may be about 1, 2, 3, 6, 10, 15, or 20 mg/kg, or an amount in a range of two of these amounts. The dose may comprise 1-20 mg/kg of the anti-CTLA-4 antibody. The dose may be 50-1000 mg of the anti-CTLA-4 antibody. The body weight of the subject may be at least 30 kg.

The anti-CTLA-4 antibody may be administered periodically, each administration for which one of the foregoing doses is administered to the subject. At each cycle of dosing, the dose may be different from a previous dose. The dosing may involve escalating doses. In one example, the anti-CTLA-4 antibody is administered about every 1, 2, 3, 4, 5, or 6 weeks. In particular, the anti-CTLA-4 antibody is administered about every 3 weeks. When describing the period of a dosing cycle, “about” may mean±1, 2, or 3 days.

In one example, the dose of the anti-CTLA-4 antibody is 10 mg/kg. The anti-CTLA-4 antibody may be administered in a dosing regimen comprising 10 mg/kg for two doses, followed by 1-6 mg/kg for extended dosing (that is, each subsequent dose is 1-6 mg/kg). The extended dosing may comprise administering a dose of 3 mg/kg or 6 mg/kg. In one example, each administration is once about every 3 weeks. In one example, the anti-CTLA-4 antibody is administered once every about 4 weeks. The dosing may take place over a period of about 3, 6, 9, 12, 15, 18, 21, 24, 27, 30, 33, 36, 39, 42, 45, 48, 51, or 52 weeks, or within a range thereof.

A flat dose of the anti-CTLA-4 antibody may be administered, which may comprise about 200 mg (~1.5 mg/kg each), 400 mg (~3 mg/kg each), 600 mg (~4.5 mg/kg each), 800 mg (~6.0 mg/kg each), or 1000 mg (~7.5 mg/kg each) of the antibody, or an amount in a range of two of these amounts.

### 5. Methods of Treatment

The antibody compositions described herein may be used to upregulate immune responses. Up-modulation of the immune system is particularly desirable in the treatment of cancers and chronic infections, and thus the antibody compositions described herein have utility in the treatment of such disorders. As used herein, the term “cancer” refers to a neoplasm or tumor resulting from abnormal uncontrolled growth of cells. “Cancer” explicitly includes leukemias and lymphomas. The term “cancer” also refers to a disease involving cells that have the potential to metastasize to distal sites.

An antibody composition described herein may be used in the manufacture of a medicament. The composition may also be administered to a subject in need of treatment. The subject may be a human. The subject may be in need of treatment of a disease or condition described herein.

The cancer may be a carcinoma, including that of the bladder, breast, colon, kidney, liver, lung, ovary, pancreas, stomach, cervix, head and neck squamous cell carcinoma (HNSCC), endometrial, renal cell, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions of the invention. Such cancers may include, but are not limited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented by the methods and compositions disclosed herein, including of the ovary, bladder, esophagus, gastrointestinal region, liver, bile duct, adenoids, breast, colon, lung, skin, pancreas, or uterus. In other specific embodiments, sarcoma, melanoma, or leukemia is treated or prevented by the methods and compositions of the invention.

In one example, the cancer is melanoma, non-small cell lung carcinoma (NSCLC), HNSCC, ovarian cancer, endometrial carcinoma, cervical cancer, renal cell carcinoma, bladder cancer, esophageal cancer, gastric cancer, gastroesophageal (GE) junction cancer, colorectal cancer, anal cancer, hepatocellular carcinoma, cancer of a bile duct, adenoid cystic carcinoma (ACC), or triple negative breast cancer (TNBC). In a further example, the NSCLC is PD-1-resistant. The ovarian cancer may be high grade serous ovarian carcinoma, which may be primary peritoneal cancer or fallopian tube cancer. The TNBC may be PD-1 resistant. The melanoma may be immuno-oncology (IO)-resistant.

In one example the cancer is a solid tumor. The solid tumor may have been one or more of histologically confirmed and cytologically confirmed. The tumor may be in a subject who has progressive locally advanced or metastatic disease. The subject may have exhibited failure or intolerance to at least one established standard medical anti-cancer therapy. The subject's cancer diagnosis may be determined by a standard of practice, which may be National Comprehensive Cancer Network (NCCN) Clinical Practice Guidelines in Oncology.

The antibody compositions and antigen binding fragments thereof may be used with another anti-tumor therapy, which may be selected from but not limited to, current standard and experimental chemotherapies, hormonal therapies, biological therapies, immunotherapies, radiation therapies, and surgery. In some embodiments, a composition described herein may be administered in combination with a therapeutically or prophylactically effective amount of one or more agents, therapeutic antibodies or other agents known to those skilled in the art for the treatment or prevention of cancer, autoimmune disease, infectious disease or intoxication. Such agents include for example, any of the above-discussed biological response modifiers, cytotoxins, antimetabolites, alkylating agents, antibiotics, anti-mitotic agents, or immunotherapeutics.

The antibody compositions and antigen binding fragments thereof may be used with another anti-tumor immunotherapy. In such an embodiment, the composition is administered in combination with a molecule that disrupts or enhances alternative immunomodulatory pathways (such as PD-1, TIM3, TIM4, OX40, CD40, GITR, 4-1-BB, B7-H1, B7-H3, B7-H4, LIGHT, BTLA, ICOS, CD27 or LAG3) or modulates the activity of effecter molecules such as cytokines (e.g., IL-4, IL-7, IL-10, IL-12, IL-15, IL-17, GF-beta, IFNg, Flt3, BLys) and chemokines (e.g., CCL21) in order to enhance the immunomodulatory effects. Specific embodiments include a bi-specific antibody comprising an anti-CTLA-4 antibody described herein or antigen binding fragment thereof, in combination with anti-PD-1, anti-B7-H1 (atezolizumab (TECENTRIQ) or durvalumab (IMFINZI)), anti-B7-H3, anti-B7-H4, anti-LIGHT, anti-LAG3, anti-TIM3, anti-TIM4 anti-CD40, anti-OX40, anti-GITR, anti-BTLA, anti-CD27, anti-ICOS or anti-4-1BB. In yet another embodiment, an antibody of the invention or antigen binding fragment thereof is administered in combination with a molecule that activates different stages or aspects of the immune response in order to achieve a broader immune response, such as IDO inhibitors.

### 6. Production

The anti-CTLA-4 antibodies described herein and antigen binding fragments thereof may be prepared using a eukaryotic expression system. The expression system may entail expression from a vector in mammalian cells, such as Chinese Hamster Ovary (CHO) cells. The antibodies may also be produced from a stable cell line that expresses the antibody from a vector or a portion of a vector that has been integrated into the cellular genome.

The anti-CTLA-4 antibodies described herein and antigen binding fragments thereof can be purified using, for example, chromatographic methods such as affinity chromatography, ion exchange chromatography, hydrophobic interaction chromatography, DEAE ion exchange, gel filtration, and hydroxylapatite chromatography. In some embodiments, fusion proteins can be engineered to contain an additional domain containing an amino acid sequence that allows the polypeptides to be captured onto an affinity matrix. For example, the antibodies described herein comprising the Fc region of an immunoglobulin domain can be isolated from cell culture supernatant or a cytoplasmic extract using a protein A or protein G column. In addition, a tag such as c-myc, hemagglutinin, polyhistidine, or Flag™ (Kodak) can be used to aid antibody purification. Such tags can be inserted anywhere within the polypeptide sequence, including at either the carboxyl or amino terminus. Other fusions that can be useful include enzymes that aid in the detection of the polypeptide, such as alkaline phosphatase. Immunoaffinity chromatography also can be used to purify polypeptides.

The present invention has multiple aspects, illustrated by the following non-limiting examples.

### Example 1

Ipilimumab Variants with Reduced CTLA-4 Binding Activity at Low pH

This example demonstrates Ipilimumab variants that have reduced CTLA-4 binding activity at low pH. Based on previous studies, anti-CTLA-4 antibodies that bind tightly inside cellular compartments and lead to lysosomal degradation of CTLA-4 can cause sustained loss of surface CTLA-4, which exacerbates irAE. This type of antibody is called a “pH insensitive antibody” (pHIA) because it maintains binding affinity with CTLA-4 at the low pH found in intracellular compartments and allows antigen-antibody complexes to be delivered to lysosomes for degradation. Data show that the clinical antibody, Ipilimumab, is a pHIA, which explains its propensity to causing irAE. Meanwhile, by preferring CTLA-4 downregulation, Ipilimumab is less effective in triggering ADCC, intratumor regulatory T-cell depletion and rejection of large established tumors.

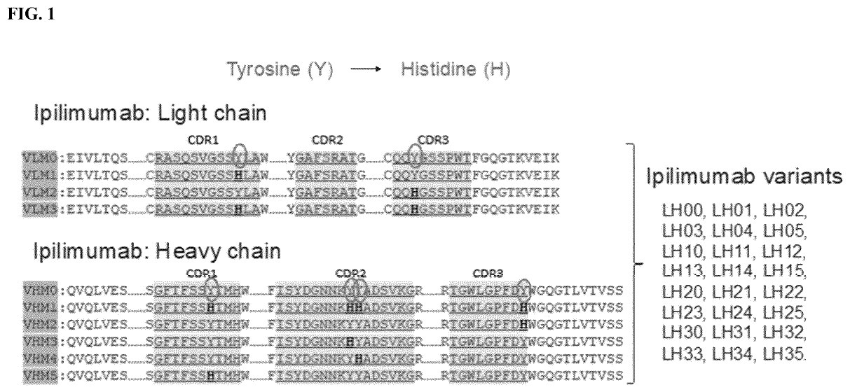

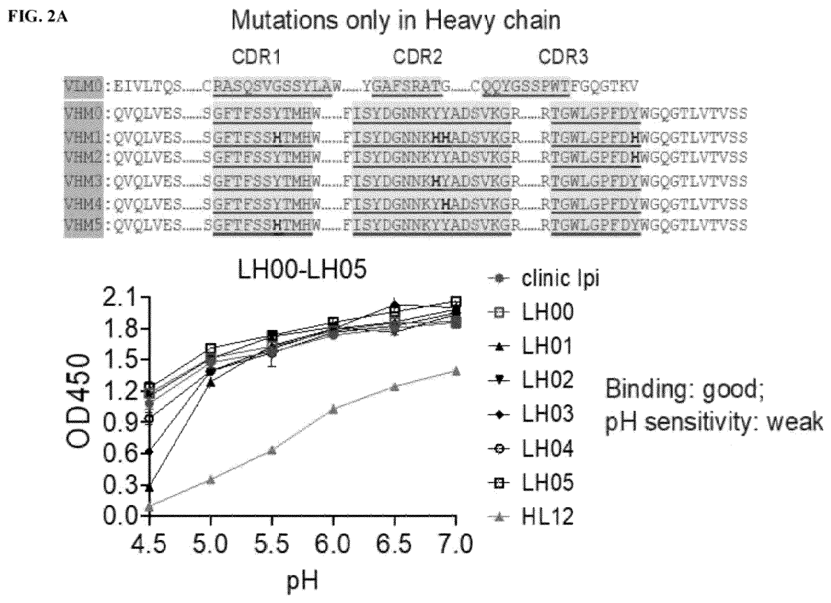

The inventors' goal was to reduce the irAE and improve the therapeutic efficacy of Ipilimumab by changing the pH sensitivity of its binding with CTLA-4. Based on the inventors' previous work, they had the insight to replace tyrosine (Y) with histidine (H) in the complementarity determining regions (CDR) of the antibody to generate pH-sensitive variants. The side chain of tyrosine will not ionize under physiological conditions, but the imidazole side chain in histidine has a pKa of approximately 6.0. Thus, below a pH of 6, which is characteristic of endocytic vesicles, the imidazole ring is mostly protonated which was hypothesized to change the CDR binding properties. pH-sensitive variants of Ipilimumab were screened by applying a similar strategy of amino acid replacements. Different combinations of Y to H mutations were designed for all of the CDR Y residues in Ipilimumab to generate a total of 23 Ipilimumab variants. The structures of these are shown in FIG. 1 and described in Table 6.

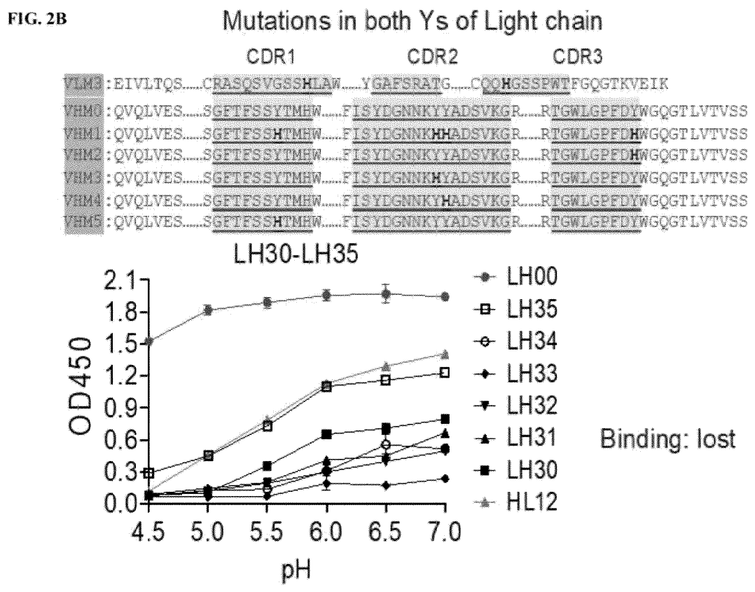

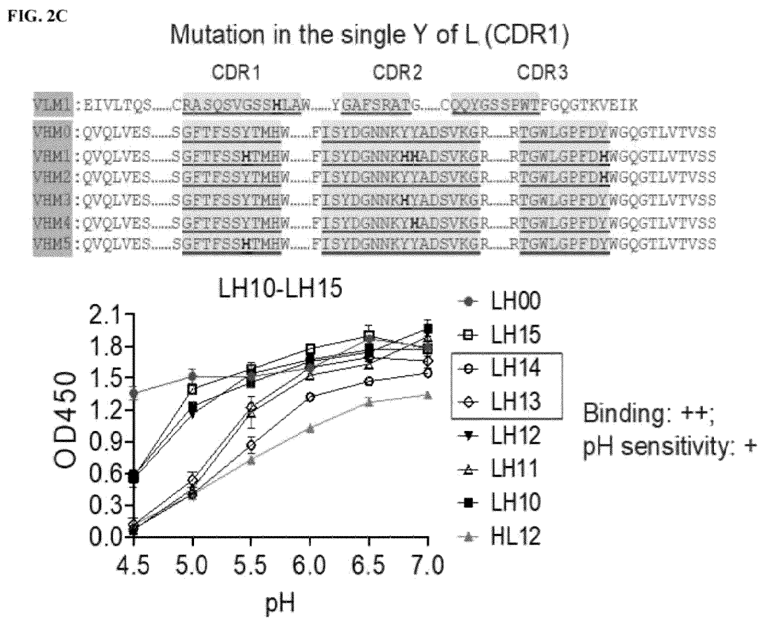

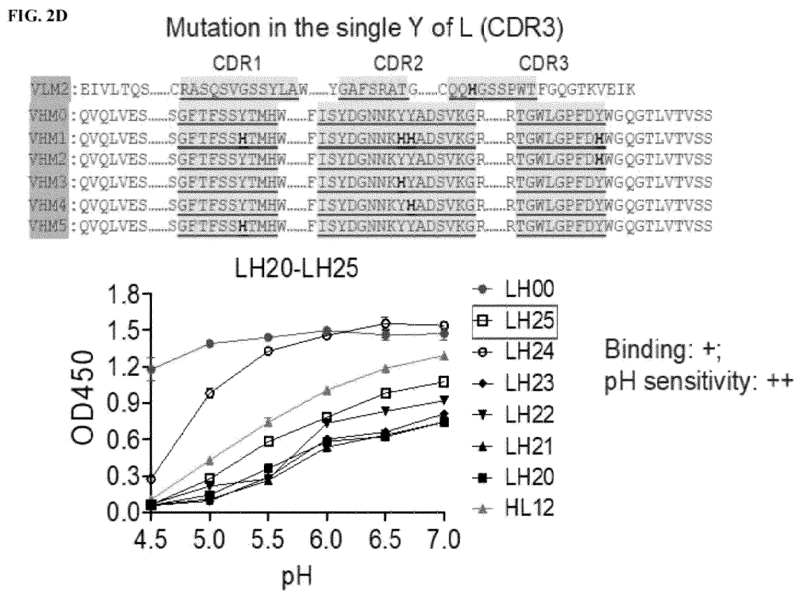

The pH sensitivity of binding of the variant antibodies to CTLA-4 compared with native Ipilimumab (LH00) was investigated by ELISA (FIG. 2). The first set of variants were generated by introducing Y to H substitutions only in the heavy chain CDR (FIG. 2A). HL12, which is a pH-sensitive anti-CTLA-4 antibody (pHSA), was used as a standard. Clinical Ipilimumab and research agent Ipilimumab (LH00, generated along with the variants) were used as pH-insensitive anti-CTLA-4 antibody controls. Five variants (LH01-05) exhibited comparable binding ability with Ipilimumab at neutral pH and did not achieve a significant improvement in pH-sensitive binding with CTLA-4 in acidic pH conditions (FIG. 2A). To improve this, two Ys in the light chain CDRs of these variants were also mutated into Hs (LH30-35) leading to significant increases of pH sensitivity, however, these variants now dramatically lost the binding affinity of CTLA-4 at neutral pH (FIG. 2B). These data suggest that at least one of the Ys in light chain CDRs is essential for the ligand-binding affinity of Ipilimumab at neutral pH. To balance the effects of pH sensitivity and binding affinity in neutral conditions, single Y to H mutations in the light chain CDRs of Ipilimumab were combined with different Y to H mutations in heavy chain CDRs (FIGS. 2C and 2D). Certain variants (LH10-12, LH15 and LH24) kept comparable binding ability with wild-type or clinical Ipilimumab at neutral pH but did not achieve a significant improvement in pH-sensitive binding with CTLA-4 in acidic pH conditions. Other variants, including LH13, LH14 and LH25, significantly increased pH sensitivity without reducing the binding of CTLA-4 at neutral pH (FIGS. 2C and 2D, highlighted by red frames).

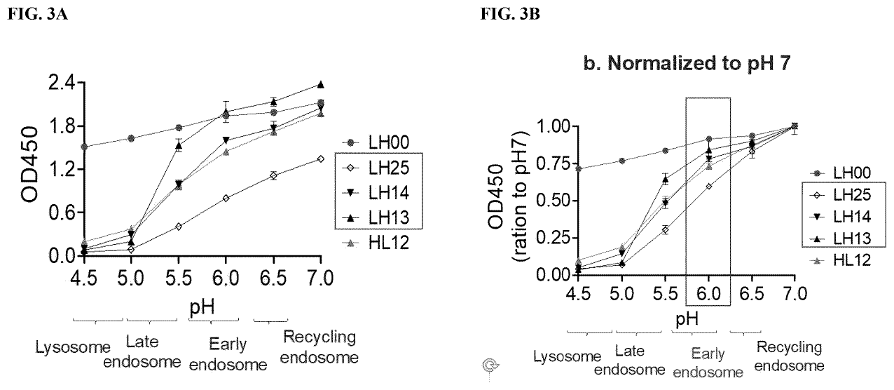

The improvement of the pH sensitivity of these 3 variants was confirmed by comparing them head-to-head with Ipilimumab in the same plate by ELISA. Data showed that below pH 5.5, which is characteristic of late endosomes and lysosomes, these 3 variants have markedly decreased binding with CTLA-4. But at approximately pH 6.0 and above, found in early endosomes, two variants, LH13 and LH14, maintained binding ability, but LH25 dissociated from CTLA-4 (FIG. 3). Thus, it was concluded that LH25 is a pH 6.0-sensitive variant while LH13 and LH14 are not. The properties of these two types of variants will be further analyzed.

### Example 2

a pH-Sensitive Ipilimumab Variants Dissociate from CTLA-4 after Endocytosis and do not Reduce CTLA-4 at the Cell Surface

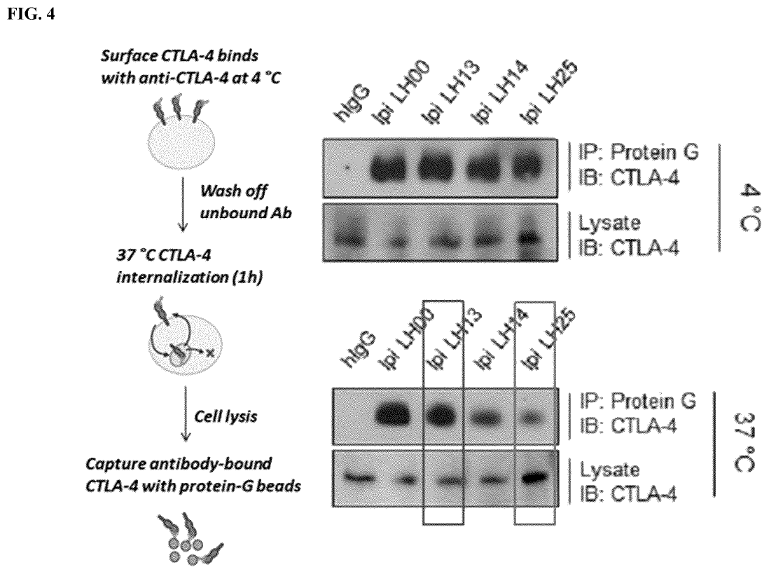

This example demonstrates an Ipilimumab variant that exhibits reduced binding to CTLA-4 at acidic pH. To determine if the pH sensitivity of Ipilimumab variants causes their dissociation from CTLA-4 in live cells, the antibodies were incubated with 293T cells stably expressing human CTLA-4 at 4° C. for 30 min. After the unbound antibodies were washed away, cells were switched to 37° C. for 1 hour. After lysis with cell lysis buffer, the anti-CTLA-4 antibodies were pulled down by protein G beads. As shown in the top panel of FIG. 4, a comparable amount of CTLA-4 was bound by wild-type and the other variants. After incubating at 37° C., much less CTLA-4 was associated with variant LH25 compared to either LH00 (wild-type Ipilimumab) or variants LH13 and LH14, which were less sensitive (but not completely insensitive) to dissociating at pH 6.0 (FIG. 4). Since LH13 and LH14 are both less sensitive to binding at pH 6.0 (referred to herein as “pH-6.0-insensitive variants”), LH13 was selected as a prototype of a pH 6.0-insensitive antibody for further study (although it does exhibit pH sensitivity).

The binding affinity of these variants to CTLA-4 were compared at pH 6.0 and pH 7.4 by surface plasma resonance (SPR). At pH 7.4, although both LH13 and LH25 exhibited somewhat reduced affinity to CTLA-4 compared with Ipilimumab, all antibodies showed comparable binding to CTLA-4 (FIG. 5, Top panel). However, when the was reduced pH to 6.0, compared to LH13, LH25 manifested a decreased affinity with a dramatically faster off-rate, suggesting that LH25 might permit CTLA-4 recycling instead of lysosomal degradation (FIG. 5, bottom panel). Data in FIGS. 4 and 5 suggest that a pH-6.0-sensitive variant would be expected to dissociate from CTLA-4 when the antibody-CTLA-4 complex is internalized inside the cells into endosomes and would release CTLA-4 more easily to traffic back to the plasma membrane allowing normal surface expression and immune regulatory activity.

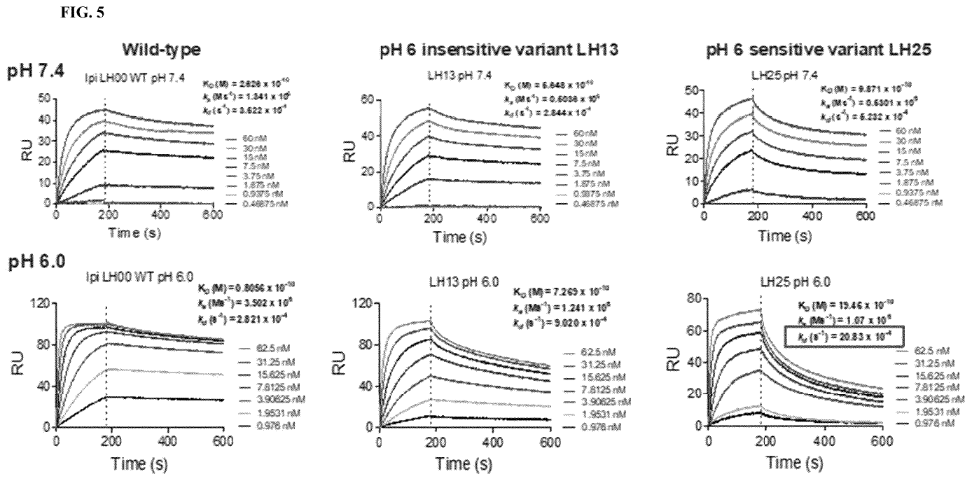

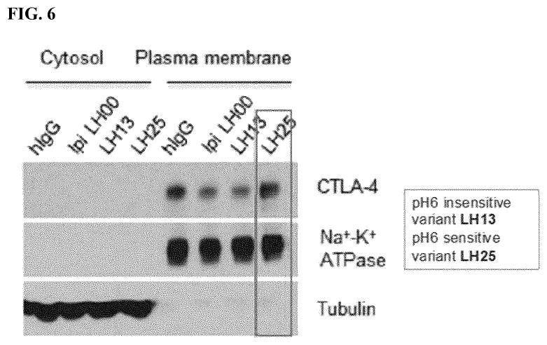

To confirm this discovery, plasma membrane-associated CTLA-4 levels following treatment of Ipilimumab or its variants were evaluated by immunoblot of cellular fractions. As shown in FIG. 6, a significant reduction of CTLA-4 was induced by wild-type Ipilimumab and its pH-6.0-insensitive variant LH13. Remarkably, pH-6.0-sensitive variant LH25 permitted Ipilimumab-induced CTLA-4 down-regulation but then allowed recovery of surface CTLA-4. These data are consistent with the hypothesis that pH-sensitivity at pH 6 is a good indicator of the longevity of antibody-mediated reduction of CTLA-4.

### Example 3

pH-Sensitive Ipilimumab Variants Rescue CTLA-4 from Lysosomal Degradation

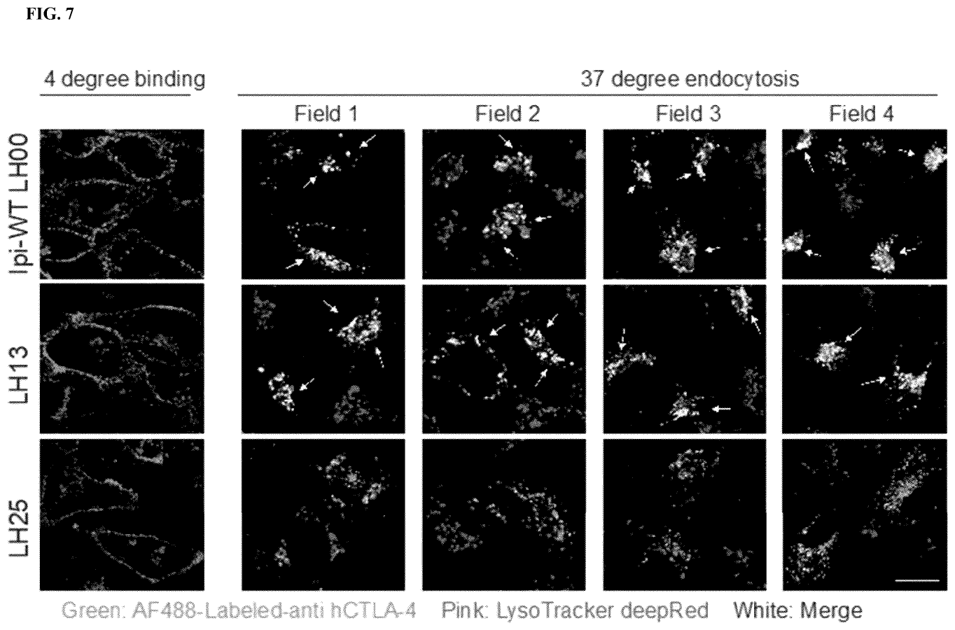

Previous studies have indicated that pH-insensitive target binding by antibodies triggers lysosomal degradation of CTLA-4, which correlates with the lack of surface CTLA-4 and irAE. To test whether the Ipilimumab variant with improved pH sensitivity rescues CTLA-4 from lysosomal degradation, anti-CTLA-4 antibodies were labeled with AF488 and incubated with CTLA-4-expressing CHO cells at 4° C., and then unbound antibody was washed away. As shown in the left panel of FIG. 7, all antibodies uniformly labeled cell surface CTLA-4 (green). After CHO cells were switched to 37° C., all anti-CTLA-4 antibodies were also internalized. However, the internalized antibodies displayed different destinations inside the cells. Both cell surface-bound wild-type Ipilimumab and the pH-6.0-insensitive variant LH13 colocalized with lysotracker indication lysosomal deposition of the antibody (FIG. 7, right panels). By contrast, pH-6.0-sensitive variant LH25 did not localize to the lysosome (FIG. 7, right panels).

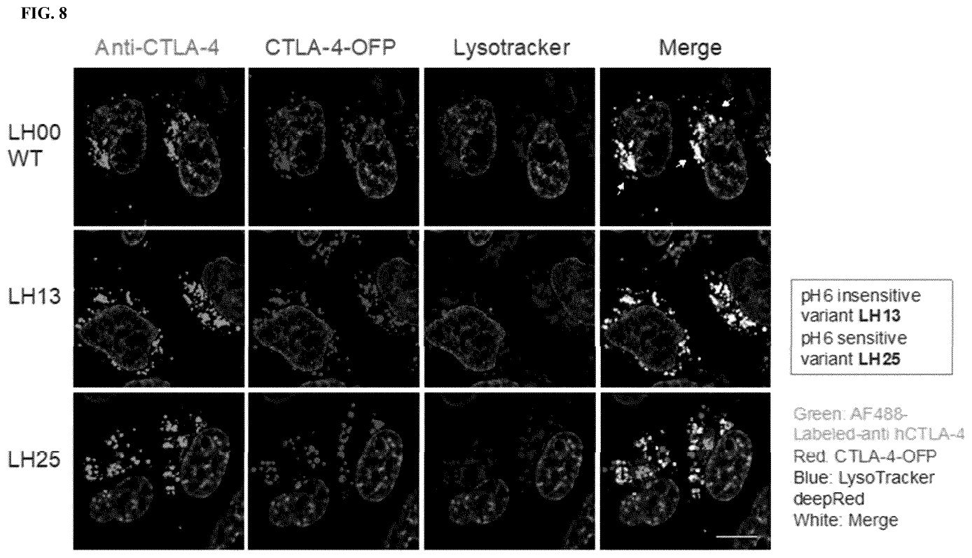

Because the CTLA-4 expressed by the CHO cells had an orange fluorescent protein (OFP) tag, it was possible to simultaneously follow the destination of antibodies and CTLA-4 intracellularly. As shown in FIG. 8, both wild-type Ipilimumab and LH13 were colocalized with CTLA-4 and lysotracker (white arrows), suggesting that they were both continuously associated with and likely promoted targeting of CTLA-4 molecules to the lysosomes. By contrast, the majority of LH25-containing vesicles were devoid of CTLA-4, indicating that LH25 dissociates from CTLA-4 after endocytosis to avoid lysosomal deposition. These data suggest that while a pH-6.0-insensitive Ipilimumab variants remain bound to CTLA-4 and apparently drive CTLA-4 into lysosomes, pH-6.0-sensitive variants dissociate from CTLA-4 after endocytosis and escape lysosomal targeting.

### Example 4

pH 6.0-Sensitive Ipilimumab Variants are Less Toxic than Ipilimumab

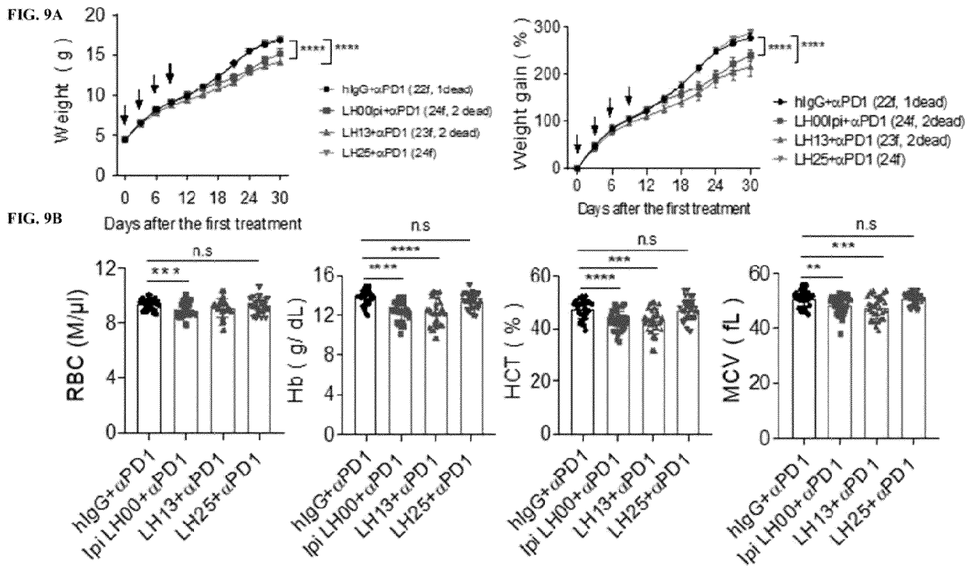

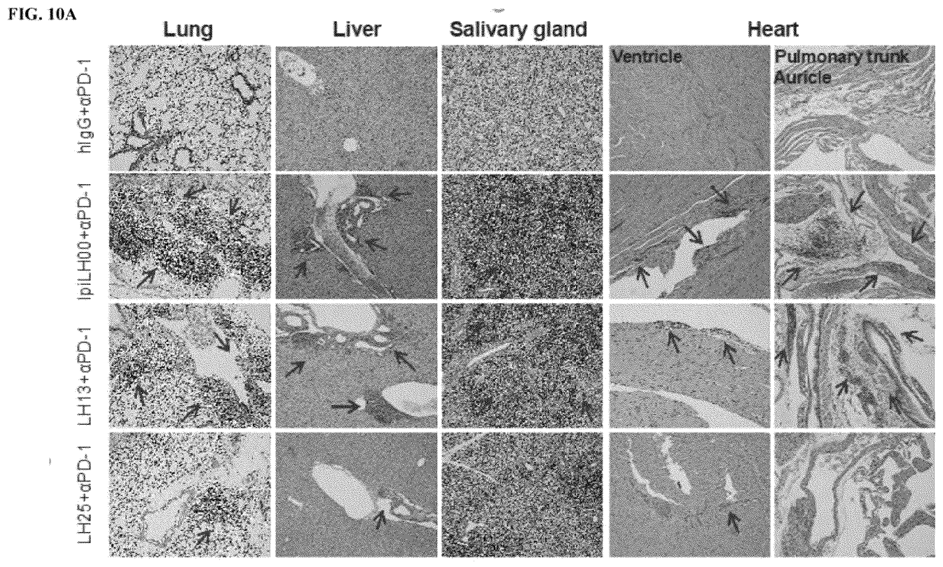

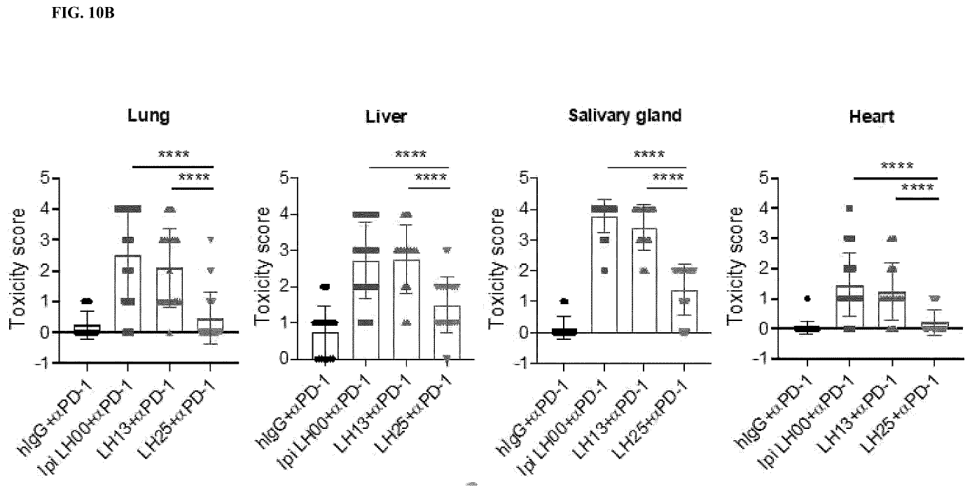

To evaluate the impact of pH sensitivity on the antibody-induced irAE, Ipilimumab, the pH-6.0 insensitive variant LH13, and the pH-6.0-sensitive variant LH25 were compared in young Ctla4^{h/h} mice (humanized CTLA-4 knock-in mice) that also received anti-PD-1 treatment to sensitize them to irAE. The mice were treated on days 10, 13, 16 and 19 after birth, and were evaluated for the body weight gain over time, hematologic changes, and histopathology alterations after 30 days of treatment (FIGS. 9 and 10). As shown in FIG. 9A, whereas mice showed substantial and statistically significant growth retardation in response to anti-PD-1+Ipilimumab or anti-PD-1+LH13, no growth retardation was observed when mice received anti-PD-1+LH25.

To study the impact of pH sensitivity on anemia, total blood cell counts were carried out at 1 month after the initiation of combination therapy (FIG. 9B). As expected, a significant reduction of blood hematocrit (HCT), total hemoglobin (Hb) and mean corpuscular volume (MCV) were observed in the majority of the mice treated with Ipilimumab+anti-PD-1 or LH13+anti-PD-1, while those that received LH25+anti-PD-1 showed no such effects (FIG. 9B). Red blood cell counts (RBC) were also largely normal in LH25+anti-PD-1 group, while it they were decreased in both Ipilimumab+anti-PD-1 and LH13+anti-PD-1 groups, although the reduction in the LH13+anti-PD-1 group was not statistically significant (FIG. 9B). These data indicate that the pH-6.0-sensitive variant, but not the pH 6.0-insensitive variant, does not cause anemia which is observed with Ipilimumab plus anti-PD1 combination therapy.

To quantitatively analyze the impact of Ipilimumab variants on tissue destruction, histological analyses of internal organs and glands from mice in the experiments in FIG. 9 were performed. Organs and glands (lung, heart and liver and salivary gland) were fixed in 10% formalin, sectioned and stained with hematoxylin and eosin (H & E), and scored in a double masked (blinded) fashion. Representative tissue sections are shown in FIG. 10A; scores from individual mice in each group are presented in FIG. 10B. As shown in FIG. 10, when combined with anti-PD-1, both wild-type Ipilimumab and its variant LH13 caused inflammation in all mice, and severe inflammation was found in all major organs. Remarkably, the inflammation in mice receiving the variant LH25 plus anti-PD-1 treatment was substantially abrogated. According to the scores from each organ, it is clear that Ipilimumab or LH13+anti-PD-1-induced much worse inflammation than the LH25+anti-PD-1 treatment (FIG. 10B). Taken together, these studies demonstrate that treatment employing the pH-6.0-sensitive variant, but not the pH-6.0-insensitive variant, alleviates Ipilimumab-induced irAE.

### Example 5

Increased Intratumor Treg Depletion and Anti-Tumor Activities of pH-6-Sensitive Ipilimumab Variants

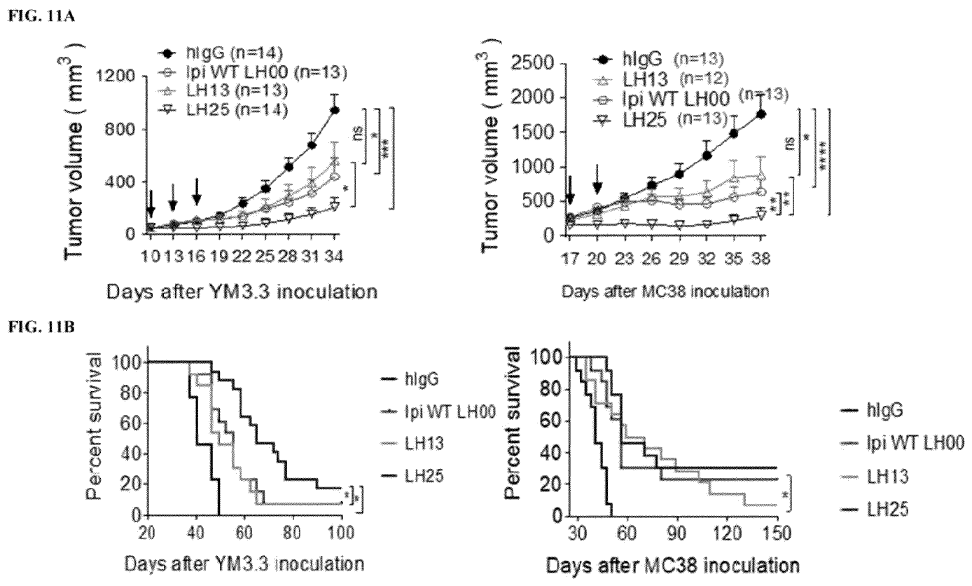

This example demonstrates that pH-sensitive Ipilimumab variants increase intratumor Treg depletion and anti-tumor activity as compared to Ipilimumab. To test if pH-sensitive Ipilimumab variants are effective in causing tumor rejection, Ctla4^{h}/h mice (humanized CTLA-4 knock-in mice) were subcutaneously inoculated with two different cancer cell models: YM3.3 melanoma and MC38 colon tumor cells. In the YM3.3 melanoma model, mice were treated with 3 doses of 1.5 mg/kg/dose of Ipilimumab, LH13, LH25 or IgG control on days 10, 13 and day 16 after tumor inoculation. The same treatments were conducted in the MC38 colon cancer model with 2 doses of antibodies on day 17 and day 20 after tumor inoculation. Tumor growth was observed by measuring tumor size every three days. As shown in FIG. 11A, at these limiting doses, all the antibodies caused retardation of tumor growth in both models.

While the reduction of tumor growth in LH13 group was not significantly different from the IgG control group, LH25 was significantly more effective in shrinking tumors compared to either wild-type Ipilimumab and LH13. Using the criterion that a mouse is considered “dead” when the diameter of the tumor reaches 2 cm, the survival curves of tumor-bearing mice treated with different antibodies were further analyzed. As shown in FIG. 11B, while LH13 displayed the same survival rate as wild-type Ipilimumab, the survival time of mice receiving LH25 was significantly longer in both melanoma and colon cancer models.

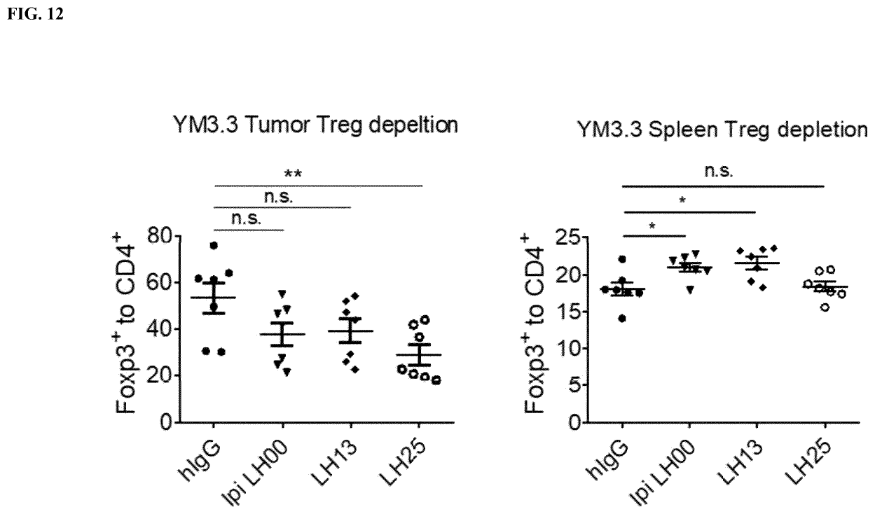

It has been demonstrated that selective depletion of tumor Tregs by anti-CTLA-4 antibodies is key to the anti-tumor effects of anti-CTLA-4 antibodies in vivo. Therefore, the effects of pH-sensitive Ipilimumab variants on intratumoral Treg depletion were assessed. Antibodies were injected into mice that were challenged with YM3.3 tumors. At 14 days, pH-sensitive variant LH25 significantly depleted intratumoral Treg, but not Treg from the spleen (FIG. 12). Wild-type Ipilimumab and LH13 were apparently less effective in tumor Treg depletion than LH25. These data reveal that pH-6.0-sensitive variants, but not the pH 6.0 insensitive variants, may improve the therapeutic effect of Ipilimumab by leading to great Treg depletion.

In summary, the data disclosed herein demonstrate that antibodies that retain binding to CTLA-4 at acidic pH 6.0 contribute to irAE. This is because a pH-6.0-insensitive antibody variant retains CTLA-4 binding leading to lysosomal targeting degradation of CTLA-4. The resulting loss of CTLA-4 surface expression apparently promotes the unregulated immune responses that lead to irAE. By contrast, a pH-6.0-sensitive Ipilimumab facilitates recycling of CTLA-4 to the cell surface and prevents its lysosomal degradation, thereby reducing the propensity to irAE. At the same time, a pH-6.0-sensitive variant described herein also triggers better anti-tumor efficacy by more efficiently depleting Tregs in the tumor than the other antibodies tested. In conclusion, these examples demonstrate that pH 6.0 sensitivity of anti-human CTLA-4 antibodies is associated with improved immunotherapeutic efficacy together with attenuated adverse inflammatory effects.

1. An anti-CTLA-4 antibody comprising:
(a) a light chain variable region comprising a complementarity determining region (CDR) 1 comprising the sequence set forth in SEQ ID NO: 1, a CDR2 comprising the sequence set forth in SEQ ID NO: 3, and a CDR3 comprising the sequence set forth in SEQ ID NO: 5; and a heavy chain variable region comprising a CDR1 comprising the sequence set forth in SEQ ID NO: 7, a CDR2 comprising the sequence set forth in SEQ ID NO: 8, and a CDR3 comprising the sequence set forth in SEQ ID NO: 12;
(b) a light chain variable region comprising a CDR1 comprising the sequence set forth in SEQ ID NO: 2, a CDR2 comprising the sequence set forth in SEQ ID NO: 3, and a CDR3 comprising the sequence set forth in SEQ ID NO: 4; and a heavy chain variable region comprising a CDR1 comprising the sequence set forth in SEQ ID NO: 6, a CDR2 comprising the sequence set forth in SEQ ID NO: 10, and a CDR3 comprising the sequence set forth in SEQ ID NO: 12; or
(c) a light chain variable region comprising a CDR1 comprising the sequence set forth in SEQ ID NO: 2, a CDR2 comprising the sequence set forth in SEQ ID NO: 3, and a CDR3 comprising the sequence set forth in SEQ ID NO: 4; and a heavy chain variable region comprising a CDR1 comprising the sequence set forth in SEQ ID NO: 6, a CDR2 comprising the sequence set forth in SEQ ID NO: 1, and a CDR3 comprising the sequence set forth in SEQ ID NO: 12.

2. The anti-CTLA-4 antibody of claim 1, wherein the light chain variable region comprises the complementarity determining region (CDR) 1 comprising the sequence set forth in SEQ ID NO: 1, the CDR2 comprising the sequence set forth in SEQ ID NO: 3, and the CDR3 comprising the sequence set forth in SEQ ID NO: 5; and the heavy chain variable region comprises the CDR1 comprising the sequence set forth in SEQ ID NO: 7, the CDR2 comprising the sequence set forth in SEQ ID NO: 8, and the CDR3 comprising the sequence set forth in SEQ ID NO: 12.

3. The anti-CTLA-4 antibody of claim 2, wherein the light chain variable region comprises the sequence set forth in SEQ ID NO: 16 and the heavy chain variable region comprises the sequence set forth in SEQ ID NO: 28.

4. The anti-CTLA-4 antibody of claim 3, comprising a light chain comprising the sequence set forth in SEQ ID NO: 21 and a heavy chain comprising the sequence set forth in SEQ ID NO: 35.

5. The anti-CTLA-4 antibody of claim 1, wherein the light chain variable region comprises the CDR1 comprising the sequence set forth in SEQ ID NO: 2, the CDR2 comprising the sequence set forth in SEQ ID NO: 3, and the CDR3 comprising the sequence set forth in SEQ ID NO: 4; and the heavy chain variable region comprises the CDR1 comprising the sequence set forth in SEQ ID NO: 6, the CDR2 comprising the sequence set forth in SEQ ID NO: 10, and the CDR3 comprising the sequence set forth in SEQ ID NO: 12.

6. The anti-CTLA-4 antibody of claim 5, wherein the light chain variable region comprises the sequence set forth in SEQ ID NO: 15 and the heavy chain variable region comprises the sequence set forth in SEQ ID NO: 26.

7. The anti-CTLA-4 antibody of claim 6, comprising a light chain comprising the sequence set forth in SEQ ID NO: 20 and a heavy chain comprising the sequence set forth in SEQ ID NO: 33.

8. The anti-CTLA-4 antibody of claim 1, wherein the light chain variable region comprises the CDR1 comprising the sequence set forth in SEQ ID NO: 2, the CDR2 comprising the sequence set forth in SEQ ID NO: 3, and the CDR3 comprising the sequence set forth in SEQ ID NO: 4; and the heavy chain variable region comprises the CDR1 comprising the sequence set forth in SEQ ID NO: 6, the CDR2 comprising the sequence set forth in SEQ ID NO: 11, and the CDR3 comprising the sequence set forth in SEQ ID NO: 12.

9. The anti-CTLA-4 antibody of claim 5, wherein the light chain variable region comprises the sequence set forth in SEQ ID NO: 15 and the heavy chain variable region comprises the sequence set forth in SEQ ID NO: 27.

10. The anti-CTLA-4 antibody of claim 6, comprising a light chain comprising the sequence set forth in SEQ ID NO: 20 and a heavy chain comprising the sequence set forth in SEQ ID NO: 34.

11. (canceled)

12. (canceled)

13. A composition comprising the anti-CTLA-4 antibody of claim 1 and a pharmaceutically acceptable excipient.

14. The composition of claim 13, comprising 20 mM histidine buffer, 8.8% (w/v) α, α-trehalose dihydrate, 0.06% (w/v) PS80, and 0.2 mM EDTA-2Na-2H₂O, wherein the composition has a pH of about 6.0.

15. The composition of claim 13, further comprising an anti-PD-1 antibody.

16. The composition of claim 15, wherein the anti-PD-1 antibody comprises a light chain comprising the sequence set forth in SEQ ID NO: 36 and a heavy chain comprising the sequence set forth in SEQ ID NO: 37.

17. A method of treating cancer in a subject in need thereof, comprising administering to the subject the anti-CTLA-4 antibody of claim 1.

18. The method of claim 17, wherein the cancer is selected from the group consisting of melanoma, non-small cell lung carcinoma (NSCLC), HNSCC, ovarian cancer, endometrial carcinoma, cervical cancer, renal cell carcinoma, bladder cancer, esophageal cancer, gastric cancer, gastroesophageal (GE) junction cancer, colorectal cancer, anal cancer, hepatocellular carcinoma, cancer of a bile duct, adenoid cystic carcinoma (ACC), and triple negative breast cancer (TNBC).

19. The method of claim 17, wherein the cancer is a solid tumor.

20. The method of claim 17, wherein the anti-CTLA-4 antibody or the composition is administered intravenously.

21. The method of claim 17, comprising administering up to 10 mg/kg of the anti-CTLA-4 antibody to the subject.

22. The method of claim 21, wherein the administration is once every 3 weeks.

23.-40. (canceled)


SEQUENCE LISTING (40 sequences)

SEQ ID NO: 1 - protein, 12 aa, synthetic construct
RASQSVGSSYLA

SEQ ID NO: 2 - protein, 12 aa, synthetic construct
RASQSVGSSHLA

SEQ ID NO: 3 - protein, 7 aa, synthetic construct
GAFSRAT

SEQ ID NO: 4 - protein, 9 aa, synthetic construct
QQYGSSPWT

SEQ ID NO: 5 - protein, 9 aa, synthetic construct
QQHGSSPWT

SEQ ID NO: 6 - protein, 10 aa, synthetic construct
GFTFSSYTMH

SEQ ID NO: 7 - protein, 10 aa, synthetic construct
GFTFSSHTMH

SEQ ID NO: 8 - protein, 16 aa, synthetic construct
ISYDGNNKYYADSVKG

SEQ ID NO: 9 - protein, 16 aa, synthetic construct
ISYDGNNKHHADSVKG

SEQ ID NO: 10 - protein, 16 aa, synthetic construct
ISYDGNNKHYADSVKG

SEQ ID NO: 11 - protein, 16 aa, synthetic construct
ISYDGNNKYHADSVKG

SEQ ID NO: 12 - protein, 9 aa, synthetic construct
TGWLGPFDY

SEQ ID NO: 13 - protein, 9 aa, synthetic construct
TGWLGPFDH

SEQ ID NO: 14 - protein, 108 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 15 - protein, 108 aa, synthetic construct
EIVLTQSPGTLSLSPGERATLSCRASQSVGSSHLAWYQQKPGQAPRLLIYGAFSRATGIPDRFSGSGSGTDFTLTISRLEPEDFAVYYCQQYGSSPWTFGQGTKVEIK

SEQ ID NO: 16 - protein, 108 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQHGSSPWTFGQGTKVEIK

SEQ ID NO: 17 - protein, 108 aa, synthetic construct
EIVLTQSPGTLSLSPGERATLSCRASQSVGSSHLAWYQQKPGQAPRLLIYGAFSRATGIPDRFSGSGSGTDFTLTISRLEPEDFAVYYCQQHGSSPWTFGQGTKVEIK

SEQ ID NO: 18 - protein, 107 aa, synthetic construct [sequence omitted]

SEQ ID NO: 19 - protein, 215 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 215 aa, synthetic construct
EIVLTQSPGTLSLSPGERATLSCRASQSVGSSHLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 215 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQHGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 215 aa, synthetic construct
EIVLTQSPGTLSLSPGERATLSCRASQSVGSSHLAWYQQKPGQAPRLLIYGAFSRATGIPDRFSGSGSGTDFTLTISRLEPEDFAVYYCQQHGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 118 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 24 - protein, 118 aa, synthetic construct
QVQLVESGGGVVQPGRSLRLSCAASGFTFSSHTMHWVRQAPGKGLEWVTFISYDGNNKHHADSVKGRFTISRDNSKNTLYLQMNSLRAEDTAIYYCARTGWLGPFDHWGQGTLVTVSS

SEQ ID NO: 25 - protein, 118 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDHWGQGTLVTVSS

SEQ ID NO: 26 - protein, 118 aa, synthetic construct
QVQLVESGGGVVQPGRSLRLSCAASGFTFSSYTMHWVRQAPGKGLEWVTFISYDGNNKHYADSVKGRFTISRDNSKNTLYLQMNSLRAEDTAIYYCARTGWLGPFDYWGQGTLVTVSS

SEQ ID NO: 27 - protein, 118 aa, synthetic construct
QVQLVESGGGVVQPGRSLRLSCAASGFTFSSYTMHWVRQAPGKGLEWVTFISYDGNNKYHADSVKGRFTISRDNSKNTLYLQMNSLRAEDTAIYYCARTGWLGPFDYWGQGTLVTVSS

SEQ ID NO: 28 - protein, 118 aa, synthetic construct
QVQLVESGGGVVQPGRSLRLSCAASGFTFSSHTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 29 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 448 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 448 aa, synthetic construct
QVQLVESGGGVVQPGRSLRLSCAASGFTFSSHTMHWVRQAPGKGLEWVTFISYDGNNKHHADSVKGRFTISRDNSKNTLYLQMNSLRAEDTAIYYCARTGWLGPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 448 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 448 aa, synthetic construct
QVQLVESGGGVVQPGRSLRLSCAASGFTFSSYTMHWVRQAPGKGLEWVTFISYDGNNKHYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 448 aa, synthetic construct
QVQLVESGGGVVQPGRSLRLSCAASGFTFSSYTMHWVRQAPGKGLEWVTFISYDGNNKYHADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 448 aa, synthetic construct
QVQLVESGGGVVQPGRSLRLSCAASGFTFSSHTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 212 aa, synthetic construct
DIQLTQSPSFLSASVGDRVTITCKASQDAGSAVAWYQQKPGKAPKLLIYWASTRHTGVPSRFSGSGSGTEFTLTISSLQPEDFATYYCQQYSSYPWTFGGGTKLEIKRTVAAPSVFIFPPSDEQLKSGTASVVCLLNNFYPREAKVQWKVDNALQSGNSQESVTEQDSKDSTYSLSSTLTKADYEKHKVYACEVTHQGLSSPVTKSFNRGEC

SEQ ID NO: 37 - protein, 445 aa, synthetic construct
EVQLVESGGGLVQPGGSLRLSCAASGFTFSRYDMSWVRQAPGKGLEWVSTISGGGRYTYYPDSVKGRFTISRDNSKNTLYLQMNSLRAEDTAVYYCTSPYGNYGMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG